(19) United States
(12) Patent Application Publication
(10) Pub. No.: US 2023/0190315 A1
(43) Pub. Date: Jun. 22, 2023

(54) VIBRATION SYSTEM AND DEVICE FOR EXTRACTION OF A LEAD OF A CARDIAC IMPLANTABLE ELECTRONIC DEVICE

(71) Applicant: XTRAC O.S LTD, Raanana (IL)

(72) Inventor: Uri STEIN, Ramat Gan (IL)

(21) Appl. No.: 17/924,861

(22) PCT Filed: May 19, 2021
(86) PCT No.: PCT/IL2021/050579

§ 371 Date: Nov. 11, 2022

(30) Foreign Application Priority Data
May 19, 2020 (IL) 274764

(51) Int. Cl. A61B 17/22

(52) U.S. Cl. CPC A61B 17/22012; A61B 17/22031; A61B 2017/22035

(57) ABSTRACT

The present disclosure provides a lead locking device that is configured to be inserted into a lumen of a lead of a cardiac implantable electronic device (CIED), such as a lead of a pacemaker, and being locked at a selected position along the lumen for being retained there for allowing extraction of the lead and/or applying vibrations to the selected position. The lead locking device includes a deformable element that may deform by application of force thereon and the deformation thereof results in radial expansion thereof such that if it is disposed within a lumen of a lead, it presses against the walls of the lumen and applies force that retains the lead locking device in position. The deformable element may be configured for reversible deformation, namely that upon removal of the force that is applied thereon, it is reversed to a contracted, non-engaging state where its radial dimension is lower than the dimension of the lumen. The deformable element may be in the form of a metallic braid, a flexible rod or a wire, an inflatable member, a spring, a structural weakening portion in a body member, such as cuts or holes, allowing a portion of the body to expand, etc.

### TECHNOLOGICAL FIELD

The present disclosure is in the field of medical-assisting equipment for extraction of a cardiac implantable electronic device, such as a lead of a pacemaker.

### BACKGROUND

Pacemaker or any implantable cardioverter-defibrillator (ICD) leads are fed into the heart through a large vein and connect the pacemaker to the implantation site of an electrode that terminates the lead which is implanted in the heart. Sometimes these inserted leads need to be removed due to one or more reasons including infection, malfunction, lead degradation, pacing system upgrade, or venous occlusion/stenosis.

Ideally (if the lead has been implanted for a short time) it should be possible to remove the lead by simple traction, however this is typically not the case. Lead removal is usually complicated by the lead's attachments to the patient's body at various places in the pathway from controller device to heart muscle, since the human body tends to incorporate foreign objects into tissue. These tissue growths (binding sites) thus hold the lead and pulling on the lead to remove it may actually endanger the patient by resulting in perforation of the heart or vein wall or tearing of the binding tissue.

In these cases the most common method of removal uses a cutting device which threads over the lead and is moved along the lead to remove any tissue attachments with a cutting tube, cutting lasers or other cutting methods. These cutting sheath or laser sheath solutions also cause problems since the tissue that is dislodged by the sheath tends to build up in front of the sheath eventually clogging the pathway that the sheath was supposed to clear.

Another optional method uses a device for simply pulling or pushing the lead for separating it from the tissue. This method can cause damaging for the tissue.

Another option is to leave the existing lead in position and insert a new lead but this is not a preferred solution as the unused lead provides additional obstruction to blood flow and heart valve function and may become infected.

Thus, there is an urgent need for an alternative solution for cardiac lead removal that significantly eases the process of lead removal and reduces the risk to patients.

### GENERAL DESCRIPTION

The present disclosure provides a lead locking device that is configured to be inserted into a lumen of a lead of a cardiac implantable electronic device (CIED), such as a lead of a pacemaker, and being locked at a selected position along the lumen for being retained there for allowing extraction of the lead and/or applying vibrations to the selected position. The lead locking device includes a deformable element that may deform by application of force thereon and the deformation thereof results in radial expansion thereof such that if it is disposed within a lumen of a lead, it presses against the walls of the lumen and applies force that retains the lead locking device in position. The deformable element may be configured for reversible deformation, namely that upon removal of the force that is applied thereon, it is reversed to a contracted, non-engaging state where its radial dimension is lower than the dimension of the lumen. The deformable element may be in the form of a metallic braid, a flexible rod or a wire, an inflatable member, a spring, a structural weakening portion in a body member, such as cuts or holes, allowing a portion of the body to expand, etc.

Therefore, an aspect of the present disclosure provides a lead locking device, i.e. a locking stylet. The lead locking device includes a deformable element disposed between a first and second rigid, non-deformable members or coupled to them, the first member is disposed at a distal end of the device and the second member is disposed at a proximal end of the device that is intended to be exposed to the physician during operation. The first and second members, and the deformable element when it is in a non-engaging state, are sized to fit into a lumen of a pacemaker's lead. Namely, their radial dimension is smaller than the radial dimension of the lumen of the lead of the pacemaker. The lead locking device includes a gripping element, or are defined at a proximal end of the first member, the gripping element or said area is having a gripping portion for gripping the device. The gripping portion also serves for the transmission of vibrations to the lead locking device and therefore to the lead. The deformable element is configured to undergo a deformation from a non-expanded and non-engaging state to an expanded, lead-engaging state, in which the lead locking device is retained in position within the lumen of the lead of the pacemaker, upon application of contraction force thereon by one or both of the first and second members.

In some embodiments of the device, the gripping element extends between a proximal and distal ends such that at least a portion thereof is disposed within a lumen of the second member.

In some embodiments of the device, at least a portion of the gripping element is disposed within a lumen of the deformable element.

In some embodiments of the device, the distal end of the gripping element is attached to or integral with the first member.

In some embodiments of the device, at least one of the first and second members is configured to move, upon application of force, towards the deformable element for resulting in said deformation.

In some embodiments of the device, the deformable element is integral with at least one of the first and second members.

In some embodiments of the device, the deformable element is disposed at the vicinity of the first member, being continuous thereto. In some embodiments, the deformable element envelopes the distal end of the gripping element.

In some embodiments of the lead locking device, a part of the gripping element constitutes the deformable element. Typically, in this embodiment, the deformable element is in the form of a spring that is received within the lumen of the first member while being in a contracted state.

In some embodiments of the lead locking device, the deformable element includes at least one of: a spring, a braided metal element and a flexible rod/wire. It is to be noted that the deformable element may be either reversibly deformed or irreversibly deformed. In the embodiments where the deformable element is reversibly deformed, the lead locking device may include a state-locking mechanism for allowing the lead for maintaining in its deformed, lead-engaging state, without applying constant force on the deformable element by a user.

In some embodiments of the lead locking device, a portion of the second member constitutes the deformable element. In some embodiments, the portion of the first member that constitutes the deformable element includes a weak portion that is deformed by application of application of force thereon such that it expands to engage the walls of the lumen of the lead. For example, the weak portion is formed of holes or generally parallel cut slits in the first member, allowing the portions between each two adjacent slits to be expanded or distorted upon application of force thereon. Therefore, in some embodiments of the device, the deformable element and at least one of the first and second members are integrally formed.

In some embodiments of the device, the first member is sized to fit between the deformable element and the gripping portion.

In some embodiments of the lead locking device, the gripping element is in the form of a wire or a thread. In some embodiments, the thread comprises metal, typically stainless steel that is capable of bending to conform with the shape of the lumen of the pacemaker's lead.

In some embodiments of the device, the second member, which typically defines the diameter of the lead locking device, has a diameter lower than 1 mm, typically lower than 0.9, 0.8, 0.7, 0.6, 0.5, 0.4, 0.3, 0.2, 0.1 mm.

In some embodiments of the device, at least one of the first and second members comprises stainless steel.

Another aspect of the present disclosure provides a lead locking device. The lead locking device includes an inflatable member having a fluid inlet for allowing introduction of fluid to inflate said inflatable member. The lead locking device further includes a fluid channel, e.g. a conduit, extending between a distal and proximal ends, the distal end of the fluid conduit is coupled to the fluid inlet and the proximal end is configured for being coupled to a fluid source. The lead locking device further includes a gripping element, or area defined at a proximal end of the first member. The gripping element is having a gripping portion for gripping the device. The gripping portion also serves for the transmission of vibrations to the lead locking device and therefore to the lead. The lead locking device further includes a deformable element configured for undergoing deformation upon inflation and/or deflation of the inflatable member from a non-expanded and non-engaging state to an expanded, lead-engaging state, respectively.

In some embodiments of the lead locking device, a part of the fluid conduit constitutes said gripping element.

In some embodiments of the device, the deformable element envelopes or surrounds the inflatable member. It is to be noted, that the deformable element does not necessarily have a physical contact with the inflatable member while the later is in the non-inflated state.

The present disclosure further provides a vibration system for generating a desired vibration profile for vibrating a lead locking device that is locked and retained at a selected position within a lumen of a lead of a CIED such as a pacemaker. The vibration system includes a vibrating element that is configured for vibrating at said desired vibration profile and transmitting the vibrations to the lead locking device, e.g. by being coupled thereto. The vibration profile is characterized by at least one of the following parameters: (i) an initial set point that results in initial tension force, namely the average position of the vibrating element between two extremes of its movement, i.e. the average between the furthest forward position and the furthest backward position; (ii) an amplitude of the vibrations from said initial set point; and (iii) frequency of the vibrations. A processing circuitry of the vibration system controls and operate the vibration system for generating the desired vibration profile.

Therefore, another aspect of the present disclosure provides a vibration system. The vibration system includes a vibration generator configured for providing vibrations at a selected vibration profile with selected parameters. A vibrating element coupled or integral with the vibration generator such that the vibration generator is configured to induce the selected vibration profile in the vibrating element, i.e. the vibrating element is configured to vibrate at or about the selected vibration profile. The vibrating element is configured to be utilized for transferring the induced vibrations to a lead locking device. The system further includes a patient-engaging arrangement configured for engaging, or bearing against a patient's body upon applying vibrations on the lead locking device.

In some embodiments of the vibration system, the selected vibration profile includes at least one of: selected vibration amplitude, initial tension force, namely the force with which the lead locking device is pulled statically before the onset of the vibrations. The initial tension force may be is some embodiments the median reference point between the peak and the nadir of the selected amplitude and vibrations frequency.

In some embodiments of the vibration system, the vibrating element includes a coupling arrangement configured for coupling with or gripping an end of the lead locking device. The coupling arrangement may be in the form of a hook for coupling to an opening of the lead locking device, or the coupling arrangement may be in the form of a lead-fastener that is fastened around a portion of the lead locking device.

In some embodiments of the vibration system, the patient-engaging arrangement includes an engaging surface designed for being adapted to the contour of the engaged body portion, e.g. the shoulder, the neck or chest area of the patient, depending on the location of the blood vessel in which the lead is located.

In some embodiments of the vibration system, the patient-engaging arrangement includes a fastener for fastening the engaging surface to the patient.

In some embodiments, the vibration system further includes a processing circuitry, i.e. a control unit, configured for controlling and operating the vibration generator to provide the selected vibration profile.

In some embodiments of the vibration system, the processing circuitry is configured for (i) applying a first vibration profile, the first vibration profile includes temporal frequency-variation in a selected range of vibration frequencies, namely applying vibrations in a range of vibration frequencies over time. The amplitude of the vibrations typically remains unchanged. The processing circuitry is further configured for (ii) identifying at least one resonating frequency in the range of vibration frequencies and store it in a memory for later use. A resonating frequency typically means a frequency which provides the most significant response by the lead locking device that is being vibrated. It is typically expressed, but not necessarily, by the most intense amplitude response.

In some embodiments of the vibration system, the processing circuitry is further configured for applying a second vibration profile that comprises vibrating in or about said resonating or most responsive frequency over a certain period of time, typically ongoing period. In other words, the first vibration profile is relatively weak and is used for identifying one or more of the most responsive and effective vibrations frequencies and the second vibration profile comprises at least one of the identified vibration frequencies from the first vibration profile in a more intense vibration profile for a certain period of time that is intended for releasing the CIED's lead from body tissues attached thereto.

In some embodiments of the vibration system, the second vibration profile includes a selected initial tension and/or a selected vibration amplitude profile, while vibrating in one of the resonating frequencies. The vibration amplitude profile includes a temporal profile of varying amplitudes, e.g. at a first period of time the amplitude may be of first value and at a second period of time the amplitude may be of a second value. In some embodiments, the amplitude may be constant over time.

In some embodiments of the vibration system, the vibration amplitude of the first vibration profile are significantly lower than the vibration amplitude of the second vibration profile.

In some embodiments, the first vibration profile is characterized by a vibration amplitude lower than 20 mm, or lower than 15, 14, 13, 12, 11, 10, 9, 8, 7, 6 or 5 mm. In some embodiments, the second vibration profile comprises vibration amplitude that at least 2-folds, 3-folds, 4-folds, 5-folds, 10-folds or 15-folds greater than the amplitude of the vibrations of the first vibration profile.

Another aspect of the present disclosure provides a vibration system. The vibration system includes a vibration generator configured for providing vibrations at a selected vibration profile. The system includes a vibrating element coupled to the vibration generator, the vibration generator is configured to induce the selected vibration profile in the vibrating element, i.e. the vibrating element is configured to vibrate at said selected vibration profile. The vibrating element is configured for transferring the vibrations to a lead locking device. A processing circuitry of the system is configured for controlling and operating the vibration generator to provide the selected vibration profile.

The processing circuitry is further configured for (i) applying a first vibration profile, the first vibration profile comprises temporal frequency-variation, i.e. applying vibrations of varying frequencies over time; and (ii) identifying at least one resonating frequency based on the response of the application of said first vibration profile, namely based on the frequency-depended response of the lead locking device to the applied vibrations. The one or more identified responsive frequencies are stored in a memory.

Another aspect of the present disclosure provides a method for identifying resonating frequency of a lead locking device being locked in position within a lead of a CIED, such as a lead of a pacemaker. The method includes: (i) applying a first, temporal frequency-varying profile of vibrations to an end of said lead locking device, said frequency-varying profile comprises a range of vibration frequencies; and (ii) identifying in said range of vibration frequencies at least one resonating frequency.

In some embodiments, the method further includes applying a second vibrations profile to the end of the lead locking device, wherein said second vibration profile comprises vibrations in or about said resonating frequency for a selected period of time.

The term “about” refers to a deviation around the value, e.g. around the value of the identified resonating frequency. The deviation may be up to 5%, 10% or up to 20% from the value.

In some embodiments of the method, the selected period of time is the majority of the time duration of said second vibration profile.

In some embodiments of the method, the second vibration profile comprises a selected initial tension and/or a selected vibration amplitude profile while vibrating in said resonating frequency.

In some embodiments of the method, the vibration amplitude of the first vibration profile is significantly lower than the vibration amplitude of the second vibration profile. For example, the amplitude of the second vibration profile can be at least 10-folds, 15-folds or 20-folds greater than the amplitude of the first vibration profile, e.g. 10 mm vs 0.5 mm respectively.

In some embodiments of the method, the first vibration profile comprises vibration amplitude lower than 2 mm. Usually, the applied amplitude of the vibrations is lower than 1 mm or lower than 0.9, 0.8, 0.7, 0.6, 0.5, 0.4, 0.3, 0.2, 0.1 mm.

Another aspect of the present disclosure provides a lead locking device according to any one of the above described embodiments use in a method for extracting a lead of a pacemaker.

In some embodiments of the use, the method includes: introducing the lead locking device into a lumen of said lead; locking the lead locking device in a desired location within the lumen; vibrating the lead locking device at a desired profile; and extracting the lead locking device together with said lead of a CIED.

In some embodiments of the use, the method further includes (i) introducing the lead locking device into a lumen of said lead; (ii) locking the lead locking device in a desired location within the lumen; (iii) vibrating the lead locking device at a desired profile; (iv) extracting the lead locking device together with internal parts of the lead, such as a metallic flexible element, i.e. a spring that is part of the lead of the pacemaker that grants the lead its rigidity and flexibility and is disposed within the lumen of said lead of a CIED; (v) introducing a second lead locking device into the lumen, the same as or different than the lead locking device of (i) and locking it to a desired location; and (vi) extracting the second lead locking device together with said lead of a CIED.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B are schematic illustrations of non-limiting examples of an embodiment of the lead locking device of the present disclosure. FIG. 1A shows the lead locking device in its non-engaging state and FIG. 1B shows the lead locking device in its lead-engaging state.

FIG. 2 is a schematic illustration of a non-limiting example of an embodiment of the lead locking device according to an aspect of the present disclosure, in which the deformable element is in the form of a deformable rod or wire.

FIG. 3 is a schematic illustration of a non-limiting example of an embodiment of the lead locking device according to an aspect of the present disclosure, in which the deformable element is in the form of a spring element.

FIG. 4 is a schematic illustration of a non-limiting example of an embodiment of the lead locking device according to an aspect of the present disclosure, in which the deformable element is constituted by weak portions of the body of the of the lead locking device.

FIG. 5 is a schematic illustration of a non-limiting example of an embodiment of the lead locking device having an inflatable member according to an aspect of the present disclosure.

FIG. 6 is a schematic illustration of a non-limiting example of an embodiment of the lead locking device having an inflatable member according to an aspect of the present disclosure.

FIGS. 7A-7D are non-limiting examples of the vibration system and its association with the patient. FIGS. 7A-7B are block diagrams of different embodiments of the vibration system; FIG. 7C is an illustration showing the components forming the vibration system; FIG. 7D is an illustration showing the association of the vibration system with a patient's body.

FIGS. 8A-8B are flow diagrams of non-limiting examples of embodiments of a method according to an aspect of the present disclosure.

### DETAILED DESCRIPTION OF EMBODIMENTS

The following figures are provided to exemplify embodiments and realization of the invention of the present disclosure.

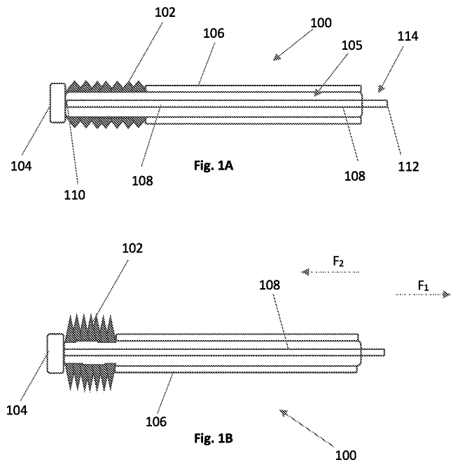

Reference is first made to FIGS. 1A-1B, which are schematic illustrations of two states of non-limiting example of a lead locking device according to an embodiment of the present disclosure. FIG. 1A shows a non-engaging state of the lead locking device 100. The lead locking device 100 includes a deformable element 102 formed of a braided material that is disposed between a first and second members 104 and 106, respectively. The dimensions of the deformable element and the first and second members in the non-engaging state are selected to fit into a lumen of a lead of a pacemaker (not shown).

The first member 104 is rigid and non-deformable and when it applies force on the deformable element 102 it causes the deformation of the later, while the first member 104 typically maintains its original shape. The second member 106 is non-contractible and has some degree of flexibility to allow its insertion along the lumen of the lead. The second member 106 can move with respect to the deformable element 102 to induce its contraction, thereby the deformable element 102 is deformed such that it engages the walls of a lead of a CIED (not shown), e.g. a pacemaker, and hold it in position due to friction forces. In other words, upon deformation of the deformable element 102, the dimensions thereof reach the dimensions of the lead's lumen and the deformable element 102 is urged against the walls of the lead as can be seen in FIG. 1B, which shows the lead locking device 100 in its engaging state.

The second member 106 is forced against the deformable member 102 in response to application of force thereon.

The lead locking device 100 includes a gripping element 108 that extends within a longitudinal lumen 105 of the lead locking device between a distal end 110 and a proximal end 112. The distal end 110 of the gripping element 108 is integral with the first member 104 and the proximal end 112 includes a gripping portion 114 allowing to grip the gripping element and applying a pulling force F₁ during application of a counter force F₂ on the second member 106 to apply force on the deformable element 102, thereby resulting in its deformation. Furthermore, the gripping portion 114 serves for coupling to a vibrating system that is configured to apply vibrations on the lead locking device.

In the figures throughout the application, like elements of different figures were given similar reference numerals shifted by the number of hundreds corresponding to the number of the respective figure. For example, element 202 in FIG. 2 serves the same function as element 102 in FIGS. 1A-1B.

FIG. 2 is a schematic illustration of a non-limiting example of another embodiment of the lead locking device of the present disclosure. The lead locking device 200 includes a deformable element 202 that is disposed between the first and second members 204 and 206 respectively. The deformable element is formed of a flexible rod or wire 216 that upon application of force by the first and/or the second members 204 or 206 is deformed from a generally straight state into a curved state, in which portions of the rod are urged to the walls of the lead's lumen (not shown) to be anchored to the walls due to the friction force applied by the rod on the walls. The gripping portion 214 of the gripping element 208 serves for gripping the lead locking device 200 to allow application of force F₁ on the deformable element 202 and to allow application of vibration profile as explained above with respect to FIGS. 1A-1B.

FIG. 3 is a schematic illustration of a non-limiting example of another embodiment of the lead locking device of the present disclosure. The lead locking device 300 includes a deformable element 302, which is in the form of a spring element 318 and is integral with the first member 304 at one end and with the second member 306 at a second end thereof. A gripping element 308 is integral with the first member 304 and extends along the entire lumen 305 that is formed in the second member 306 such that a portion thereof, namely the gripping portion 314, projects out of the proximal end 328 of the second member 306 to allow the gripping thereof by a practitioner or by a vibrating system. The gripping element 308 passes through the spring 318, namely the spring 318 confines a portion of the gripping element 308 with its loops. The spring 318 is configured such that in a non-biased state its dimensions fits to pass through the lumen of a lead of a CIED (not shown), e.g. a pacemaker, and when it is biased to a contracted state, e.g. by compression forces thereon by one or both of the first and second members 304 and 306, the dimensions thereof, in particular the radial dimension, increases such that it presses the walls of the lumen of the lead of a CIED and retains the lead locking device 300 in position by friction forces.

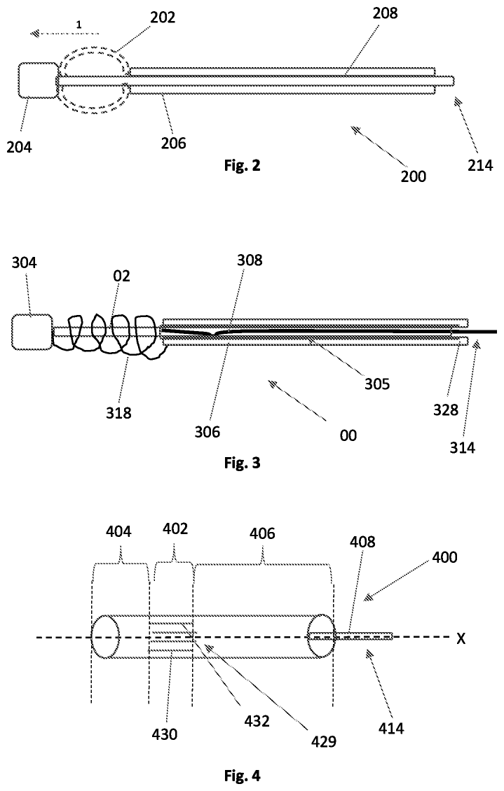

FIG. 4 is a schematic illustration of a non-limiting example of another embodiment of the lead locking device of the present disclosure. In this example, the lead locking device 400 includes a body 401 that different portions thereof constitute the first member 404, the second member 406 and the deformable element 402 that is formed between the first and second members 404 and 406. The portion that constitute the deformable element 402 is formed of weak portions 430, for example elongated axial cuts along a longitudinal axis X of the lead locking device 400. It is to be noted that the weak portions may be formed of drills, holes, decreased thickness of the body, cuts in various directions or any suitable configuration. These weak portions 430 decrease the rigidity of the of the portion of the body defining the deformable element such that by applying a force on this portion of the body, the remaining body parts 432 bend in a general radial orientation with respect to the longitudinal axis X and urge against the walls of the lumen of the lead of a pacemaker (not shown). The friction force between the remaining bended body parts 432 and the wall of the lumen of the lead retains the lead locking device 400 in position. A gripping element 408 axially extends from the first member 404 via the distal end 429 and beyond the proximal end 428 of the second member 406 for being exposed for allowing the gripping of the gripping portion 414 for (i) applying pulling force on the lead locking device, (ii) applying vibration profile of the lead locking device, or a combination thereof

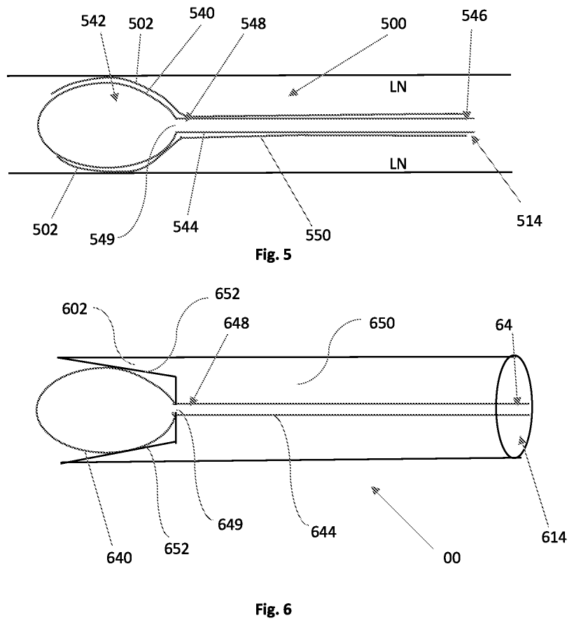

FIGS. 5-6 are another examples of non-limiting examples of embodiments of the lead locking device. These examples include an inflatable deformable element that upon inflation thereof causes the lead locking device to retain in position within the lumen of the lead of the pacemaker.

FIG. 5 shows a lead locking device 500 that includes an inflatable member 540 configured to be inflated by a fluid, e.g. gas such as air or liquid, that flows to an internal space 542 in the inflatable member. A fluid channel, e.g. a fluid conduit 544 extends between a proximal end 546 and a distal end 548. The inflatable member 540 includes a fluid inlet 549 fluidically connecting between the distal end 548 of the fluid conduit 544 and the inflatable member 540, in particular the internal space 542 of the inflatable member 540. The fluid conduit 544 includes a fluid opening for introducing fluid into the fluid conduit 544 to thereby flow towards the internal space 542 of the inflatable member 540 for inflating thereof. The inflatable member 540 is at least partially enveloped by a deformable element 502, such as a metal cover, that is configured for undergoing deformation upon inflation and/or deflation of the inflatable member 540, namely to be enlarged upon inflation of the inflatable member. Upon inflation of the inflatable member 540 the deformable element 502 is expanded and engages the lumen LN of the lead of the pacemaker and when the inflatable member 540 deflates, the deformable element 502 is contracted to non-expanded state, in which it is not forcingly engaging the lumen LN of the lead of the pacemaker. Introduction of fluid with pressure into the inflatable member 540 results in the inflation thereof In some embodiments, as exemplified in FIG. 5, the deformable element 502 may be integral with an elongated body member 550, such as a metal wire, that extends along the fluid conduit 544. The elongated body member 550 and the fluid conduit 544 extend between the inflatable member and beyond the external tip of the lead of the CIED. Therefore, the elongated body member 550 and the fluid conduit 544 are made of flexible materials that allow them to bend along the lumen LN of the lead of the pacemaker they are introduced into. The proximal end 546 of the fluid conduit 544 and respective proximal end of the elongated body member 550 constitute, each alone or together, a gripping portion 514 for gripping the lead locking device upon application of vibrations thereto.

FIG. 6 shows a lead locking device 600 that includes an inflatable member 640 having a fluid inlet 649 for allowing introduction of fluid therein to inflate the inflatable member 640. The inflatable member 640 is accommodated between walls 652 of a deformable element 602 such that upon inflation thereof, it urges against the walls 652 to thereby bend them towards the lumen of the lead of the pacemaker to engage them and apply a friction force that retain the lead locking device 600 in position within the lumen of the lead. A fluid conduit 644 is fluidically connected to the fluid inlet 649 for allowing fluids to flow therethrough to inflate the inflatable member 640 and extends between a proximal end 646 and a distal end 648, which is connected to the fluid inlet 649. The deformable element 602 is integral with an elongated body member 650, such as a metal wire that extends along the fluid conduit 644. The elongated body member 650 and the fluid conduit 644 extend between the inflatable member and beyond the external tip of the lead of the CIED. Therefore, the elongated body member 650 and the fluid conduit 644 are made of flexible materials that allow them to bend along the lumen of the lead of the pacemaker they are introduced into. The proximal end 646 of the fluid conduit 644 and respective proximal end of the enveloping body member 650 constitute, each alone or together, a gripping portion 614 for gripping the lead locking device upon application of vibrations thereto.

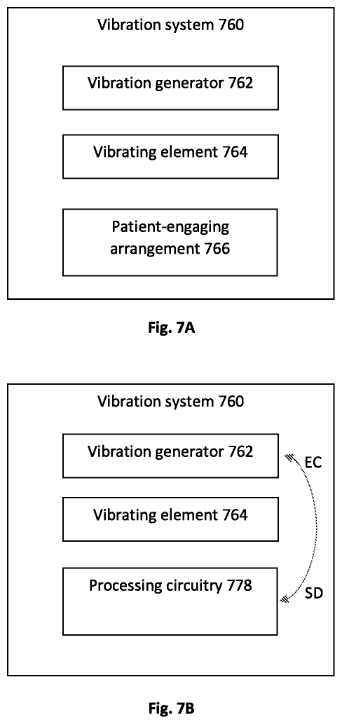

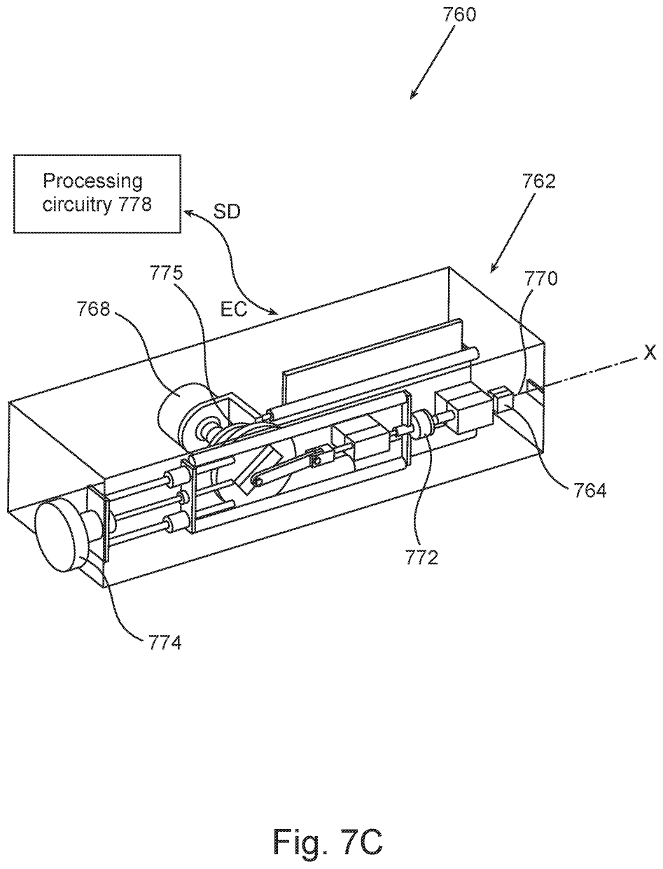

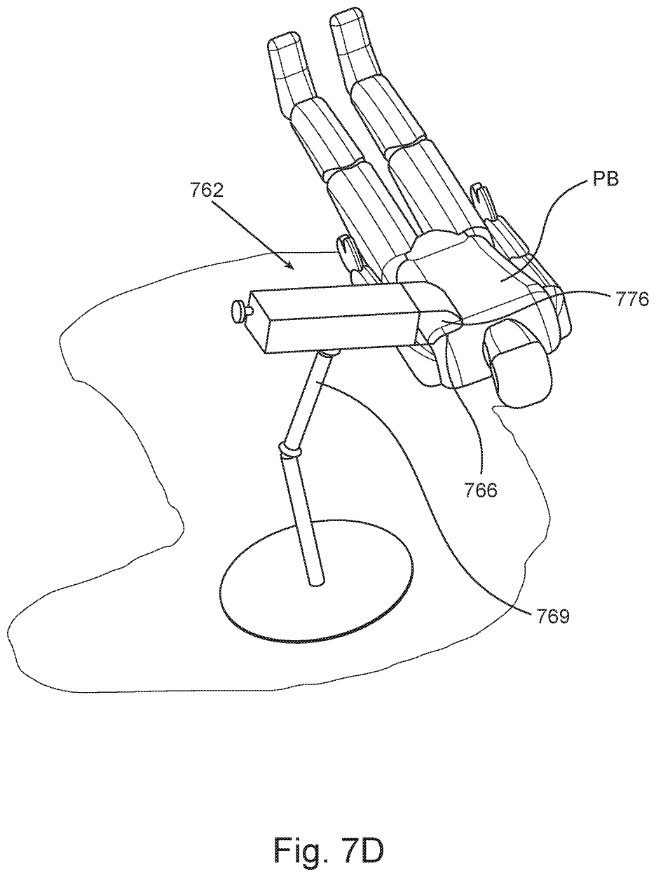

FIGS. 7A-7D are non-limiting examples of a vibration system and its engagement configuration to a subject according to an aspect of the present disclosure, FIGS. 7A-7B are block diagrams exemplifying different embodiments of the vibration system of the present disclosure, FIGS. 7C-7D are illustrations exemplifying different embodiments of the vibration system of the present disclosure. The vibration system 760 includes a vibration generator 762 configured for generating selected vibration profile to be applied on a medical component. The vibration system comprises a vibrating element 764 that is configured to be vibrated in the selected vibration profile in response to the generated vibration profile of the vibration generator 762 and transmit the vibration to the medical component. The system further includes a patient-engagement arrangement 766 that is designed for engaging patient's body PB while transmitting the vibration profile to a lead locking device locked to a lead of a CIED or directly to the lead of the CIED.

The vibrating element 764 is typically coupled to a proximal end of a lead locking device or to the top thereof and the vibration profile is transmitted via the lead locking device towards the locking position between the locking mechanism of the lead locking device and the lead of the CIED.

The vibration system comprises a motor complex 768 that is configured to vibrate at the desired vibration profile so as to induce vibration on the vibrating element 764. The motor complex 768 includes a motor that is configured to rotate at a selected profile. The motor complex 768 further includes a rod and the rotations of the motor are transformed to the desired vibration profile via said rod. The vibrating element 764 respectively vibrates in response to the vibrations produced by the motor complex 768 and is configured to axially vibrate along an axis X. The vibrating element 764 is configured to be coupled to or for gripping a gripping portion of a lead locking device (not shown) by a coupling arrangement or mechanism 770 in the vibrating element 764. The vibration system further comprises a force sensor 772 for providing a feedback of the force of the vibrations that applied to the vibrating element 764.

The vibrations are characterized by at least: (i) an initial set point that results in initial tension force, namely the average position of the vibrating element between two extremes of its movement, i.e. the average between the furthest forward position and the furthest backward position; (ii) an amplitude; and (iii) frequency. The initial set point is adjusted by an average force adjustor 774 that is configured to set the vibrating element 764 at the selected position to thereby vibrate at a desired amplitude and frequency around the selected set point.

The vibration system may further includes a zero angle sensor 775 that senses the phase shift between the rotating wheel and the force created on or by the lead locking device. The measuring of the phase is then used to identify the resonance frequency of the lead locking device.

The patient-engagement arrangement has an engagement surface 776 that is designed to fit the contour of a patient's body PB, that may be the shape of the shoulder, chest or neck, depending where the lead of the pacemaker is extracted from.

The vibration system is fastened to the patient's body PB by a fastener (not shown) for ensuring that the engagement arrangement specifically and the vibration system in general do not move and remain in position while the vibration are applied to the lead locking device that is locked to the lead of the pacemaker that is implanted in the patient. The fastener may be fastened to the arm of the patient or around the chest of the patient.

The vibrating system further comprises a processing circuitry 778 configured for controlling and operating the vibration generator 762 by transmitting it execution commands EC for providing the desired vibration profile, namely controlling at least one of the following parameters: an initial set point that results in initial tension force (may be adjusted manually by the average force adjustor 774); an amplitude; and frequency. The processing circuitry may be integral part of the vibration generator or external thereof at a remote location that is in data communication therewith and operatively connected thereto. The processing circuitry 778 may also transmit the parameters-related data to a display so as to allow the physician that performs the operation to monitor the system performance.

The processing circuitry 778 may also configured for controlling the vibration generator for identifying desired frequency for applying to the lead locking device by (i) applying a first vibration profile and (ii) identifying at least one resonating frequency according to the frequency-depended response to the application of the first vibration profile.

The first vibration profile is applied at a certain time duration and is characterized by a relatively low amplitude and/or initial tension profile and by a varying frequency profile over time. While applying the first vibration profile, the processing circuitry is configured to receive sensed data SD from the force sensor 772 and the zero angle sensor 775 indicative of the frequency response of each frequency and analyze it to identify the most intense response, namely the resonating frequency and the phase shift. The processing circuitry stores the one or more most responsive frequencies for utilizing it in the application of further vibration profiles.

The processing circuitry 778 may be further configured to apply a second vibration profile that is characterized by a frequency of or about the resonating frequency that is identified in the first vibration profile. The second vibration profile is configured for vibrating the lead locking device at a sufficient intensity to release the lead it is locked to from the tissues attached to the lead, to thereby allowing the extraction of the lead from the patient.

The second vibration profile is typically characterized by a greater amplitude and/or initial set point for a selected duration of time.

In some embodiments, as exemplified in FIG. 7D, the vibration generator 762 may be mounted on a mounting arrangement 769 that is configured to adjustably elevating, positioning and/or aligning the vibration generator 762 to the desired position with respect to the patient's body PB.

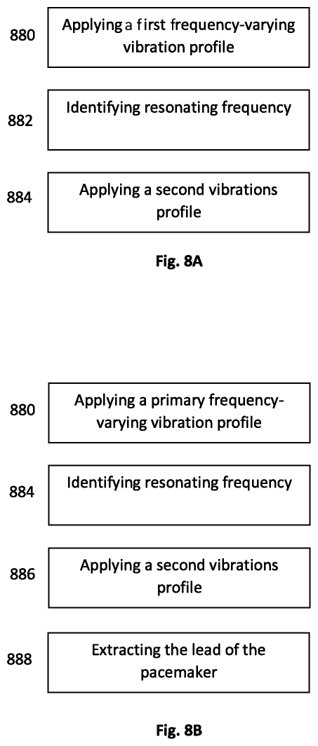

FIGS. 8A-8B are flow diagrams of non-limiting examples of a method for identifying optimal vibration frequency and using it for extracting a lead of a pacemaker according to an aspect of the present disclosure. FIG. 8A exemplifies a method that includes applying a first frequency-varying vibration profile to a lead locking device 880 to thereby vibrating a lead of a pacemaker locked thereto. The response of the lead locking device for the applied vibrations of the first vibration profile varies for each frequency and the method includes identifying one or more of the most responsive frequencies 882 from the plurality of the applied frequencies of the first vibration profile, being effectively the resonating frequency or close to the resonating frequency. The method further includes applying a second vibrations profile to the lead locking device 884, the second vibration profile is characterized by a greater amplitude and/or initial tension force, i.e. the average tension force, and is further characterized by one or more of the most responsive frequencies. Namely, the applied second vibrations profile includes vibrations at a frequency of at least one of the identified most responsive frequencies from the first frequency-varying vibrations profile for a selected duration of time.

FIG. 8B differs from FIG. 8A by including extraction of the lead of a pacemaker 886 following the application of the second vibration profile. The application of the second vibration profile releases the lead from the tissues and allow its easy extraction from the patient. In some embodiments the lead locking device may be extracted with internal parts of the lead and the lead remains within the patient. In these embodiments, the method includes introducing a second lead locking device for the extraction process. The second lead locking device is typically different than the first lead locking device, however it can also be the same. Once the second lead locking device is locked to the lead, the extraction is carried out by pulling the lead locking device together with the lead until it is extracted from the patient.

1. A lead locking device, comprising:
a deformable element disposed between a first and second members, said first and second members are sized to fit into a lumen of a cardiac implemented electronic device (CIED) lead;
a gripping element having a gripping portion for gripping the device;
wherein the deformable element is configured to undergo a deformation from a non-expanded state to an expanded, lead-engaging state upon application of force thereon by one or both of said first and second members.

2. The lead locking device of claim 1, wherein the gripping element extending between a proximal and distal ends such that at least a portion thereof is disposed within a lumen of the second member.

3. The lead locking device of claim 2, wherein the distal end of the gripping element is attached to the first member.

4. The lead locking device of claim 1, wherein at least a portion of the gripping element is disposed within a lumen of the deformable element.

5. The lead locking device of claim 1, wherein at least one of the first and second members is configured to move towards the deformable element for resulting in said deformation.

6. The lead locking device of claim 1, wherein the deformable element is integral with at least one of said first and second members.

7. The lead locking device of claim 1, wherein the deformable element is disposed at the vicinity of the first member.

8. The lead locking device of claim 1, wherein a part of the gripping element constitutes the deformable element.

9. The lead locking device of claim 1, wherein the deformable element comprises at least one of: a spring, a braided metal element and a flexible rod or wire.

10. The lead locking device of claim 1, wherein a portion of the second member constitutes the deformable element.

11. The lead locking device of claim 10, wherein the deformable element is formed of weak portions of the second member that allow other portions thereof to deform upon application of force thereon.

12. The lead locking device of claim 1, wherein the deformable element and the first member are integrally formed.

13. The lead locking device of claim 1, wherein the second member is sized to fit between the deformable element and the gripping portion.

14. The lead locking device of claim 1, wherein the gripping element is in the form of a wire.

15.-38. (canceled)

39. A lead locking device according to claim 1 for use in a method for extracting a lead of a cardiac implemented electronic device (CIED).

40. The lead locking device of claim 39, wherein the method comprises
introducing the lead locking device into a lumen of said lead;
locking the lead locking device in a desired location within the lumen;
vibrating the lead locking device at a desired profile;
extracting the lead locking device together with said lead of a CIED.

41. The lead locking device of claim 39, wherein the method comprises
(i) introducing the lead locking device into a lumen of said lead;
(ii) locking the lead locking device in a desired location within the lumen;
(iii) vibrating the lead locking device at a desired profile;
(iv) extracting the lead locking device together with internal parts of said lead of a CIED;
(v) introducing a second lead locking device into the lumen, different than the lead locking device of (i) and locking it to a desired location;
(vi) extracting the second lead locking device together with said lead of a CIED
